(19) United States
(12) Patent Application Publication
(10) Pub. No.: US 2016/0326087 A1
(43) Pub. Date: Nov. 10, 2016

(54) PROCESS FOR PREPARATION OF DIMETHYL-(E)-BUTENEDIOATE

(71) Applicant: SHILPA MIDICARE LIMITED, Raichur (IN)

(72) Inventors: RAFIUDDIN DR, RAICHUR (IN); BHAGAT RAJ PIPAL, RAICHUR (IN); RAVINDRA REDDY UMMADI, RAICHUR (IN); AKSHAY KANT CHATURVEDI, RAICHUR (IN)

(73) Assignee: SHILPA MEDICARE LIMITED, RAICHUR (IN)

(21) Appl. No.: 15/110,104

(22) PCT Filed: Oct. 18, 2014
(86) PCT No.: PCT/IB2014/065440

§ 371 Date: Jul. 7, 2016

(30) Foreign Application Priority Data
Jan. 24, 2014 (IN) 0308/CHE/2014

(51) Int. Cl. C07C 67/08; C07C 69/60; C07C 67/52

(52) U.S. Cl. CPC C07C 67/08; C07C 67/52; C07C 69/60; C07B 2200/13

(57) ABSTRACT

The present invention relates to a process for preparation of dimethyl-(E)-butenedioate(I).

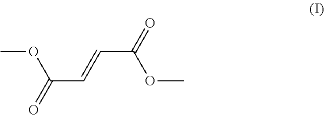

The process for preparation of dimethyl-(E)-butenedioate (I), comprises reaction of Fumaric acid with methanol in the presence of C₂-C₄alkanoyl halideas coupling catalyst.

### FIELD OF THE INVENTION

The present invention relates to a process for preparation of dimethyl-(E)-butenedioate (I).

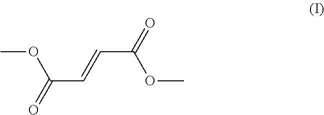

The process for preparation of dimethyl-(E)-butenedioate (I), comprises reaction of Fumaric acid with methanol in the presence of C₂-C₄ alkanoyl halide as coupling catalyst.

### INTRODUCTION

Dimethyl-(E)-butenedioate (I) is generally also known as Dimethyl Fumarate and is available commercially as TECDIFERA®, for the treatment of patients with relapsing forms of multiple sclerosis.

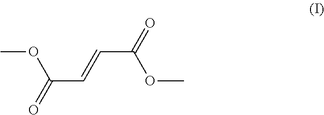

Dimethyl-(E)-butenedioate is a white to off-white powder that is highly soluble in water with a molecular mass of 144.13

Fumaric acid is an intermediate in the citric acid cycle that is hydrated by the enzyme fumarase to maleic acid. The use of fumaric acid for the treatment of psoriasis was introduced in 1959. Earlier known derivatives of fumaric acid like dihydroxy fumaric acid, fumaramide, and fumaronitrile, suffered from problem of insufficient resorbtion, due to which high doses were to be utilized leading to high toxicity and serious side effects.

Gamrath et al in U.S. Pat. No. 2,764,609 described conversion of dialkyl maleate to dialkyl fumarate by heating around 100° C. in presence of phosphorus oxychloride, thionyl chloride or phosphorus trichloride etc. and then cooling.

Further the literature article in journal Synthesis, Issue 6, Pages 316-17 (1971) provided a method of esterification of unsaturated organic acids using a boron trifluoride etherate-alcohol reagents, thereby synthesizing dimethyl-(E)-butenedioate.

U.S. Pat. No. 4,851,439, U.S. Pat. No. 4,959,389 and U.S. Pat. No. 5,424,332 provided further disclosure of fumaric acid derivatives and their pharmaceutical compositions for the treatment of psoriasis and psoriatic arthritis.

Kadowaki, Yasushi; et al. in U.S. Patent Appl. No. 2002/0002306 disclosed a method of producing dimethyl fumarate containing no catalyst residue by use of a heterogenous Group VIII catalyst.

The single-crystal structure of dimethyl fumarate is disclosed in Kooijman, H., et al, Acta Crystallographica E60:0917-0918 (2004).

Guzowski et al in WO2012170923A1 described a process for the preparation of dimethyl Fumarate, involving the esterification of fumaric acid and methanol in the presence of sulfuric acid as an acid catalyst. It is mentioned that the dimethyl fumarate obtained by the mentioned process contained no more than trace amounts of dimethyl sulfate.

Though the review of the above mentioned literature discloses diverse processes for preparation of dimethyl-(E)-butenedioate, but due to one or more reasons most of them are not particularly convenient and amenable to industrial scale-up. Thus, there is an apparent need of new improved processes for preparation of dimethyl-(E)-butenedioate, which may be efficient, cost-effective, industrially scalable and may overcome the drawbacks of various prior disclosed processes, e.g., use of corrosive chemicals in large quantity.

Therefore, inventors of the present application provide a process for preparation of dimethyl-(E)-butenedioate, which is amenable to scale up at industrial level and solves purity/compliance related issues of the end product.

### SUMMARY OF INVENTION

Particular aspects of the present specification relate to the process for the preparation of dimethyl-(E)-butenedioate (I).

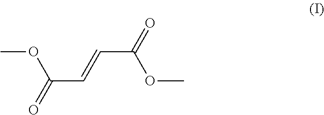

The process of preparation of dimethyl-(E)-butenedioate (I) comprises the steps of—
a) providing a solution of Fumaric acid in methanol at room temperature;
b) adding coupling catalyst C₂-C₄ alkanoyl halide to the reaction mixture;
c) raising the temperature of reaction mixture up to a range of 50-75° C.;
d) cooling the reaction mixture;
e) isolating the solid material;
f) optionally, treating the solid material obtained in step e) with an organic solvent or a mixture thereof, and recovering the crystalline solid.

One aspect of the present invention provides that crystalline dimethyl-(E)-butenedioate (I) prepared according to the process of the present invention is having HPLC purity greater than 99.95% (by HPLC) and is characterized by Monomethyl Fumarate content of 0.01% or less.

In a further aspect, the present invention relates to crystalline dimethyl-(E)-butenedioate (I), characterized by X-ray powder diffraction pattern—having at least five 2θ° peaks selected from the XRPD peak set of 9.82, 10.88, 17.44, 19.81, 23.71, 23.97, 26.17, 33.26 and 41.26±0.1°; or substantially according to FIG.-1 and DSC isotherm comprising at least one endothermic peak ranging between—

a. Peak-1—Between 100 to 110° C.; or

b. Peak-2—Between 135 to 155° C.

Another aspect of the present invention provides crystalline dimethyl-(E)-butenedioate (I), characterized by moisture content of less than 0.3% w/w (Karl Fischer Analysis), TGA weight loss of less than 0.5% w/w, up to temperature of 120° C. and particle size distribution of D90=252-280 μm.

Further particular aspects of the invention are detailed in the description part of the specification, wherever appropriate.

### BRIEF DESCRIPTION OF THE DRAWINGS

FIG. 1 is illustration of X-ray powder diffraction (“XRPD”) pattern of dimethyl-(E)-butenedioate (I) obtained in Example 1.

FIG. 2 is illustration of Differential Scanning Calorimetry (“DSC”) curve of dimethyl-(E)-butenedioate (I) obtained in Example 1.

### ABBREVIATIONS

| | |
|---|---|
| API | Active Pharmaceutical Ingredient |
| DSC | Differential Scanning Calorimetry |
| HPLC | High-Performance Liquid Chromatography |
| RT | Room Temperature |
| TGA | Thermo-Gravimetric Analysis |
| XRPD | X-Ray Powder Diffraction Pattern |

### DETAILED DESCRIPTION

As set forth herein, embodiments of the present invention relate to a process for the preparation of dimethyl-(E)-butenedioate (I),

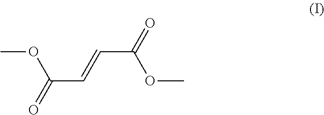

comprising the steps of:
a) providing a solution of Fumaric acid in methanol at room temperature;
b) adding coupling catalyst C₂-C₄ alkanoyl halide to the reaction mixture;
c) raising the temperature of reaction mixture up to a range of 50-75° C.;
d) cooling the reaction mixture;
e) isolating the solid material;
f) optionally, treating the solid material obtained in step e) with an organic solvent or a mixture thereof, and recovering the crystalline solid.

The individual steps of the process according to the present invention for preparing dimethyl-(E)-butenedioate (I), are detailed separately herein below.

Step a) comprises providing a solution of Fumaric acid in methanol at room temperature, wherein solution of Fumaric acid is provided in 5-10 volumes of methanol (in mL) w.r.t. weight of Fumaric acid (in g). In a particular embodiment of the present invention 50 g Fumaric acid was provided as solution in 400 mL methanol.

Step b) comprises adding coupling catalyst C₂-C₄ alkanoyl halide to the reaction mixture, wherein C₂-C₄ alkanoyl halide is added in catalytic amount ranging from 0.35-0.5 moles per 1 mole of Fumaric acid used in step a). In a preferred embodiment C₂-C₄ alkanoyl halide is selected from acetyl chloride or acetyl bromide.

Use of coupling catalyst, C₂-C₄ alkanoyl halide in the process of the present invention bears the inventive merit of avoiding the use of corrosive chemicals like Sulphuric acid and thus also negates the possibility of formation of toxic impurities like dimethyl sulphate.

Step c) comprises raising the temperature of reaction mixture up to a range of 50-75° C.;

The reaction mixture obtained from step b) is heated to temperature ranging from 50-75° C.; preferably up to 60-70° C. The raised reaction temperature is maintained for time duration of 5-15 hrs depending upon the progress of the reaction as is intermittently checked by HPLC.

Step d) comprises cooling the reaction mixture to room temperature, wherein cooling is performed in controlled manner of not more than 1° C./minute. The cooled reaction mass is further subjected to stirring for time duration varying from 1 to 3 hours.

Step e) comprises isolating the solid material, wherein the solid material separated in step d) is filtered by any method known to person having skill in the art. The solid material is then dried at room temperature for time duration ranging from 1-3 hours.

Step f) comprises optionally treating the solid material obtained in step e) with an organic solvent or a mixture thereof and recovering the crystalline solid.

Solid material obtained in step e) is optionally treated with an organic solvent or a mixture thereof. The said organic solvent may be selected from C₁-C₃ alcoholic solvent for e.g. methanol, ethanol or n-propanol. Solid material obtained in step e) is dissolved in 8-15 volume C₁-C₃ alcoholic solvent. The reaction mixture is then heated to get a clear solution. Heating is preferably performed to a temperature of 50-60° C. The clear solution is then filtered through celite bed to get a particle free solution, which is again heated if required, to get clear solution. Under continuous stirring the reaction mixture is slowly cooled to RT and then further to a temperature of 0-15° C., wherein it is maintained for time duration of 1-3 hours.

The solid material obtained above is filtered and suck dried at room temperature for time ranging from 30 mins to 1 hour. Further drying may be performed under reduced pressure conditions. Reduced pressure conditions may be suitably utilized by person skilled in the art in order to obtain the dried material. In one particular embodiment of the current application, further drying was performed under vacuum. This drying may be performed for time ranging from 2 to 4 hrs depending upon compliance to the product characteristics of dimethyl-(E)-butenedioate (I).

Process of isolating dimethyl-(E)-butenedioate (I) may further comprise processes but not limited to conventional processes including scrapping and if required filtering from slurry which may be carried out at room temperature for the suitable durations.

With increasing emphasis on safety of the pharmaceutical substances, regulatory agencies demand stringent in process mechanisms for determination and the control of DNA reactive (mutagenic) impurities/genotoxic impurities in medicinal products.

The process related impurities that appear in the impurity profile of dimethyl-(E)-butenedioate (I) may be substantially removed by the process of the present invention resulting in the formation of pure dimethyl-(E)-butenedioate (I) in a crystalline form. The merit of the process according to the present invention resides in that—product obtained after drying is highly pure and very stable and can be suitably stored for prolonged durations. Substantially pure dimethyl-(E)-butenedioate (I) obtained according to the process of the present invention results in the final API purity by HPLC of more than 99.95% w/w. The final API is characterized by Monomethyl Fumarate content of 0.01% or less.

The crystalline dimethyl-(E)-butenedioate (I), obtained according to the process of the present invention is characterized by X-ray powder diffraction pattern substantially according to FIG.-1 and DSC isotherm comprising at least one endothermic peak ranging between—

a. Peak-1—Between 100 to 110° C.; or

b. Peak-2—Between 135 to 155° C.
The characteristic peaks and the corresponding d-spacing values of the crystalline dimethyl-(E)-butenedioate (I) obtained by the process of the present invention are tabulated in the Table-1 and is designated as Form-SD.

TABLE 1

| S. No. | Angle (2θ°) ± 0.10 | d-Spacing Value (A°) |
|---|---|---|
| 1. | 9.82 | 9.004 |
| 2. | 10.88 | 8.121 |
| 3. | 17.44 | 5.081 |
| 4. | 19.81 | 4.478 |
| 5. | 21.52 | 4.126 |
| 6. | 21.76 | 4.081 |
| 7. | 21.99 | 4.039 |
| 8. | 23.71 | 3.750 |
| 9. | 23.97 | 3.709 |
| 10. | 26.17 | 3.402 |
| 11. | 27.21 | 3.274 |
| 12. | 27.32 | 3.261 |
| 13. | 33.26 | 2.691 |
| 14. | 41.26 | 2.186 |
| 15. | 44.87 | 2.018 |

Minor variations in the observed 2 θ° angles values may be expected based on the analyst person, the specific XRPD diffractometer employed and the sample preparation technique. Further possible variations may also be expected for the relative peak intensities, which may be largely affected by the non-uniformity of the particle size of the sample. Hence, identification of the exact crystalline form of a compound should be based primarily on observed 2 theta angles with lesser importance attributed to relative peak intensities. The 2 theta diffraction angles and corresponding d-spacing values account for positions of various peaks in the X-ray powder diffraction pattern. D-spacing values are calculated with observed 2 theta angles and copper K α wavelength using the Bragg equation well known to those of having skill in the art of XRPD diffractometry science.

The crystalline dimethyl-(E)butenedioate (I), obtained according to the process of the present invention is further characterized by moisture content of less than 0.3% w/w (Karl Fischer Analysis) and TGA weight loss of less than 0.5% w/w, up to temperature of 120° C.

The crystalline dimethyl-(E)-butenedioate (I), prepared according to the process of the present invention is consistently obtained with particle size, wherein D90 is greater than 250 μm. Preferably particle size of crystalline dimethyl-(E)-butenedioate (I) obtained according to the process of current invention, ranges between D90=252-280 μm. This uniform particle size distribution of the final API which is suitable for handling and further processing is achieved directly by the process of the present invention and obviates the need for any type of milling procedure to be performed. Thus process of the present invention has advantage of directly providing the material suitable for making pharmaceutical compositions, having acceptable and comparable dissolution profiles.

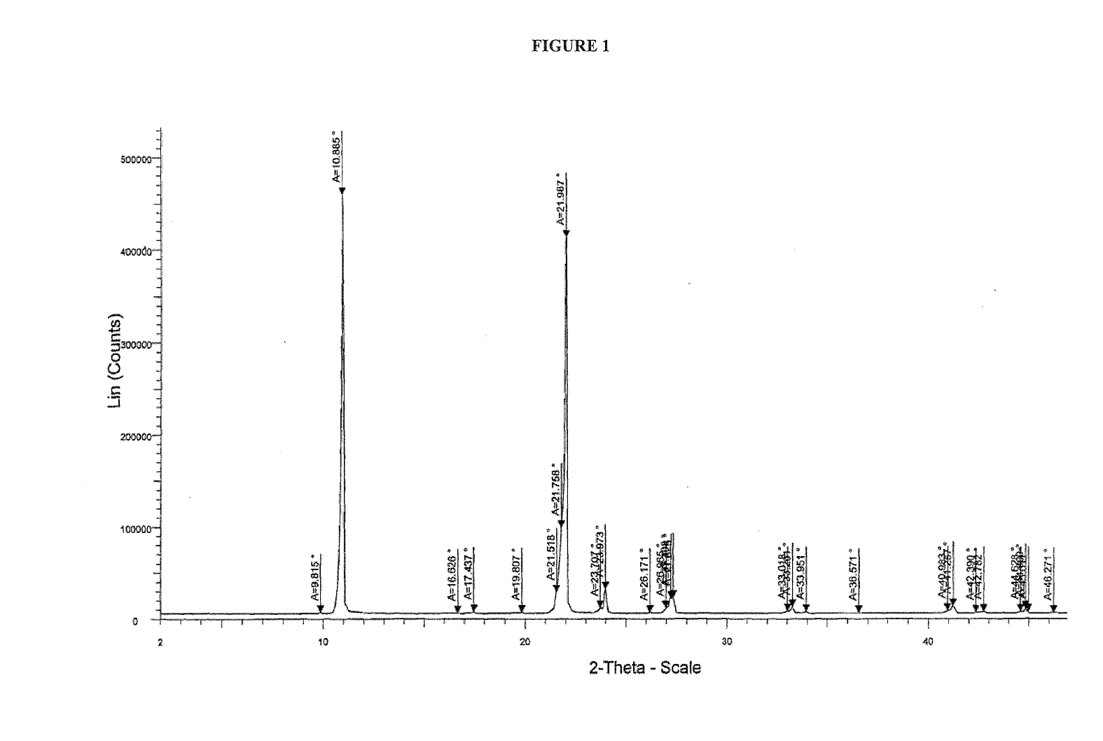

The samples of dimethyl-(E)-butenedioate (I) described herein were analyzed by XRPD on a Bruker AXS D8 Advance Diffractometer using X-ray source—Cu Kα radiation using the wavelength 1.5418 Å and lynx Eye detector. DSC was done on a Perkin Elmer Pyris 7.0 instrument. Illustrative example of analytical data for dimethyl-(E)-butenedioate (I) obtained in the examples is set forth in the FIGS. 1 & 2.

### EXAMPLES

### Example-01

### Process for Preparation of dimethyl-(E)-butenedioate (I)

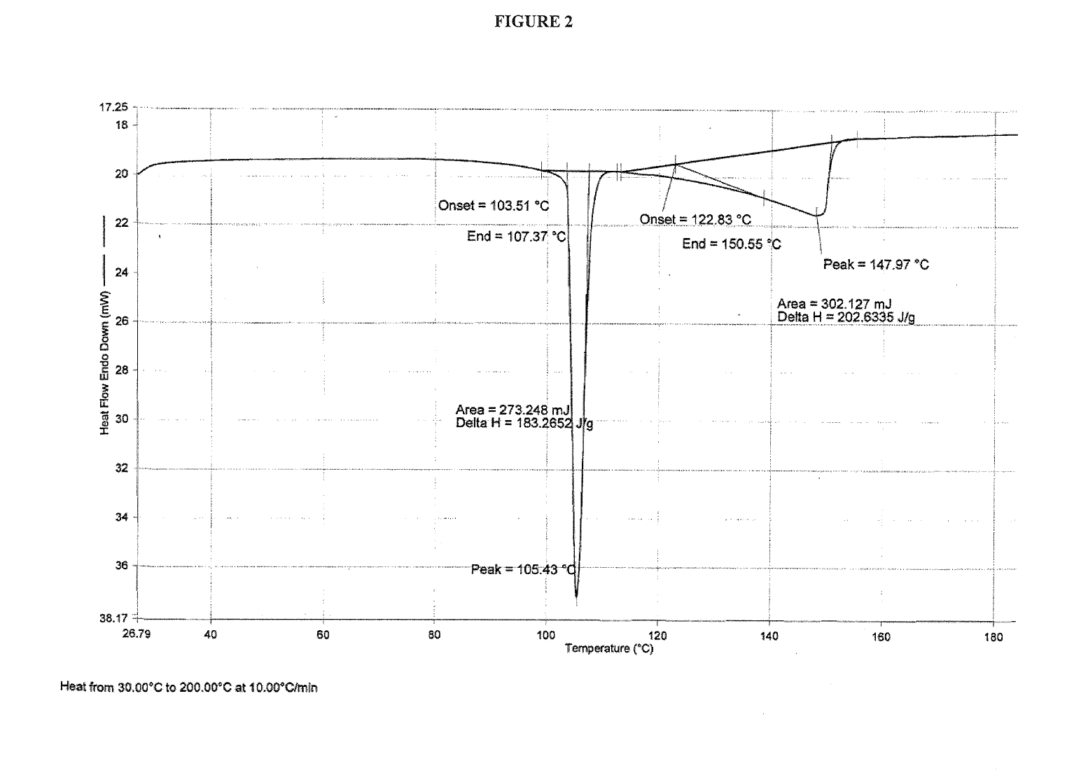

In a clean round bottom flask 50.0 g fumaric acid and 400.0 mL methanol, were charged under stirring at room temperature. Then under stirring, 14.6 g acetyl chloride was added slowly to the reaction mixture. The reaction temperature was raised to 70° C. and maintained for 12 hours, followed by cooling to room temperature and further stirring for one hour. The separated solid material was filtered and suck dried for two hours at room temperature. 45.0 g of the dried solid material was charged to 540.0 mL methanol at room temperature and then heated to ˜55° C. to get the clear solution. The clear solution was filtered through celite bed to get the particle free solution. The clear solution was again heated to ˜55° C. and then cooled to room temperature under stirring. The reaction mixture was further cooled to ˜15° C. under stirring and maintained at this temperature for 2 hours. The solid material obtained was filtered and suck dried for 30 mins at RT. The solid material was then further dried under vacuum at room temperature for three hours, to obtain 33.2 g dimethyl-(E)-butenedioate (I) in a crystalline form having the XRPD diffractogram and DSC thermogram as shown in FIGS. 1 and 2 respectively.

HPLC Purity: 99.97%

Moisture Content: 0.09% w/w

The crystalline dimethyl-(E)-butenedioate (I) obtained by this process was designated as Form-SD.

### Example-02

### Process for Preparation of dimethyl-(E)-butenedioate (I)

In a clean round bottom flask 40.0 g fumaric acid and 330.0 mL methanol, were charged under stirring at room temperature. Then under stirring, 34.0 g acetyl bromide was added slowly to the reaction mixture. The reaction temperature was raised to 65° C. and maintained for 12 hours, followed by cooling to room temperature and further stirring for one hour. The separated solid material was filtered and suck dried for two hours at room temperature. 35.0 g of the dried solid material was charged to 450.0 mL ethanol at room temperature and then heated to ˜50° C. to get the clear solution. The clear solution was filtered through celite bed to get the particle free solution. The clear solution was again heated to ˜55° C. and then cooled to room temperature under stirring. The reaction mixture was further cooled to ˜10° C. under stirring and maintained at this temperature for 2 hours. The solid material obtained was filtered and suck dried for 30 mins at RT. The solid material was then further dried under vacuum at room temperature for three hours, to obtain 26.1 g dimethyl-(E)-butenedioate (I) in a crystalline form having the XRPD diffractogram and DSC thermogram according to FIGS. 1 and 2 respectively.

HPLC Purity: 99.98%

Moisture Content: 0.15% w/w

The crystalline dimethyl-(E)-butenedioate (I) obtained by this process was designated as Form-SD.

### Example-03

### Process for Preparation of dimethyl-(E)-butenedioate (I)

In a clean round bottom flask 40.0 g fumaric acid and 330.0 mL methanol, were charged under stirring at room temperature. Then under stirring, 34.0 g acetyl bromide was added slowly to the reaction mixture. The reaction temperature was raised to 65-70° C. and maintained for 14 hours, followed by cooling to 10-15° C. and further stirring for one hour. The separated solid material was filtered and suck dried for one hour at room temperature. Dried the material for 4-5 hours under vacuum at room temperature and check the moisture content (NMT 0.50% w/w). 35.0 g of the dried solid material was charged to 450.0 mL methanol at room temperature and then heated to 60-65° C. to get the clear solution. The clear solution was filtered through hyflow bed to get the particle free solution and washed with methanol. Distilled off methanol under vacuum at 40-45° C. Cooled the reaction mixture to 0-5° C. and again stirred for 2-3 hours. Filtered the solid and dried for 1 hour at room temperature. Dried the material at 25-35° C. for 4-5 hours. The obtained dimethyl-(E)-butenedioate (I) in a crystalline form having the XRPD diffractogram and DSC thermogram according to FIGS. 1 and 2 respectively.

HPLC Purity: 99.99%

Moisture Content: 0.21% w/w

The crystalline dimethyl-(E)-butenedioate (I) obtained by this process was designated as Form-SD.

While the foregoing pages provide a detailed description of the preferred embodiments of the invention, it is to be understood that the description and examples are illustrative only of the principles of the invention and not limiting. Furthermore, as many changes can be made to the invention without departing from the scope of the invention, it is intended that all material contained herein be interpreted as illustrative of the invention and not in a limiting sense.

1. A process for the preparation of dimethyl-(E)-butenedioate(I), 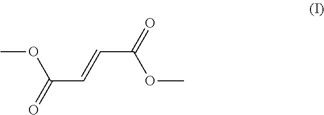
comprising the steps of:
a) providing a solution of Fumaric acid in methanol at room temperature;
b) adding coupling catalyst C₂-C₄alkanoyl halide to the reaction mixture;
c) raising the temperature of reaction mixture up to a range of 50-75° C.;
d) cooling the reaction mixture;
e) isolating the solid material; and
f) optionally treating the solid material obtained in step e) with an organic solvent or a mixture thereof, and recovering the crystalline solid.

2. A process for the preparation of dimethyl-(E)-butenedioate(I) according to claim 1, wherein, in step a) solution of Fumaric acid is provided in 5-10 volumes of methanol (in mL) w.r.t. weight of Fumaric acid (in g).

3. A process for the preparation of dimethyl-(E)-butenedioate(I) according to claim 1, wherein, coupling catalyst C₂-C₄alkanoyl halide is selected from acetyl chloride or acetyl bromide, and is used in quantity of 0.35-0.5 moles per mole of Fumaric acid.

4. A process for the preparation of dimethyl-(E)-butenedioate(I) according to claim 1, wherein, in step c) reaction mixture temperature is raised up to 60-70° C.

5. A process for the preparation of dimethyl-(E)-butenedioate(I) according to claim 1, wherein, optional step f) comprises treatment of the solid material obtained in step e) with C₁-C₃ alcoholic solvent and involves the steps of—
i. providing a solution of solid material obtained in step e) with 8-15 volume C₁-C₃ alcoholic solvent selected from methanol, ethanol or n-propanol;
ii. heating the reaction mixture to temperature of 50-60° C.;
iii. cooling the reaction mixture to a temperature of 0-15° C.; and
iv. recovering the crystalline solid.

6. (canceled)

7. Crystalline dimethyl-(E)-butenedioate(I) characterized by X-ray powder diffraction pattern-having at least five 2θ° peaks selected from the XRPD peak set of 9.82, 10.88, 17.44, 19.81, 23.71, 23.97, 26.17, 33.26 and 41.26±0.1°; or substantially according to FIG.-1 and DSC isotherm comprising at least one endothermic peak ranging between—
a. Peak-1—Between 100 to 110° C.; or
b. Peak-2—Between 135 to 155° C.

8. A process for the preparation of dimethyl-(E)-butenedioate(I) characterized by X-ray powder diffraction pattern-having at least five diffraction angle peaks (2θ°) selected from the XRPD peak set of 9.82, 10.88, 17.44, 19.81, 23.71, 23.97, 26.17, 33.26 and 41.26±0.1° and DSC isotherm comprising at least one endothermic peak ranging between 100 to 110° C. (peak-1); and 135 to 155° C. (peak-2) comprising the steps of
a) providing a solution of Fumaric acid in methanol at room temperature;
b) adding coupling catalyst C₂-C₄alkanoyl halide to the reaction mixture;
c) raising the temperature of reaction mixture up to a range of 50-75° C.;
d) cooling the reaction mixture;
e) isolating the solid material;
f) providing a solution of solid material obtained in step e) with 8-15 volume C₁-C₃ alcoholic solvent;
g) heating the reaction mixture to temperature of 50-60° C.;
h) cooling the reaction mixture to a temperature of 0-15° C.; and
i) recovering the crystalline solid.

9. Crystalline dimethyl-(E)-butenedioate(I), according to claim 8, further characterized by moisture content of less than 0.3% w/w (Karl Fischer Analysis).

10. Crystalline dimethyl-(E)-butenedioate (I) according to claim 8, further characterized by presence of Monomethyl Fumarate, to an extent of 0.01% or less (by HPLC) and particle size distribution of D90=252-280 μm.
